SEQUENCE LISTING (19 sequences)

SEQ ID NO: 1 - protein, 11 aa, synthetic construct
  REGION 1..11: note = C terminal degradation tag
AANDENYALAA

SEQ ID NO: 2 - DNA, 18 bp, synthetic construct
  misc_feature 1..18: note = I SceI cleaved base pair sequence
  source 1..18: mol_type = other DNA
tagggataacagggtaat

SEQ ID NO: 3 - DNA, 4129 bp, synthetic construct
  misc_feature 1..4129: note = pCTXVP60
  source 1..4129: mol_type = other DNA
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaggatctattaatacgactcactatagggatcccgggcgcgccgtcgactttaaagttcatccttttcagaaacatatgaaaatccattagcagttcctccaagattaaaaacaagagtagacttagatggtcttggtccaactggtctaacatcaataagttcagtaagatcaataagagtagtagaagctccagttccagcataaaaatatccatcaacagaaagtccaatttccatgaatccagaagcaaaagtaagttgccaaacaaaaaattgtcctggcataagagcagaagaataattattaagagaaagtccaatagtaactggaagtggttgagatccagttccatattgagttccattagcagatccagcagtagcattaacatctccagttcttctaacaacagaagcaaacataattggagtattctttccaactggagctgcagctggagttcctggagtagtaacaattctatctggttgtggagtataagtaatagcattagcagatggagtagaaataactccagaagccataacaaaaagtccagctggattttgagcagttccagtaacaacagcatagatagacttagcaacagtttgagatccagaagtattagtagttggttgaagatttcctggagctccagtagcaaatccaagttcataagcttgaacagtagtaacatttggagctccagaattagagttccaaatagctccaaatccaacccatccagcagctggaatacctggtccattgaatggaacaaaagacatatctggaaatccatctggagcaacttgagaaattggattatcaatagcagatccagcattagcataccaaaattgaagaacattagtagcattagatcctggataagaagcagatcctcttctatgatcgatatcagcaaatcttggagaagaccatccataagtagatccattaagattccaatgtctattacaagtagaaaatcctcctggaactggttgaagtccaacaatttgtccattccatctattatcatttccaactccagtaagaactggagtagtaagaagtccagctggatagatagaatcaacagtcttagaagatggagttctaatcataacaaattcaaaatcttcagatggtctagtttcaacagtaacttgaatagcagaagtagatcctccaaatggattaataagattattataaacagaaagaacaagagttggaacaagtcctggatctccagttggatgatacatatttggtctaaggtctggcatagtaatagtaactggttcaagagacctagcatcaataacaacatgtggaaattgtctaacttcaagacctggtccaatttcaattcctggtggaataacagcagcaacaagtcttcctccaaaaactccagatccagcaacaatgaatctgaattgcattcctccagcccatccagcatacatttgagaaagaacagcagtgaatggattattttgtggagaatgttgaacagtataaagaatagatcctggagcatcagcaacagaccaagtaaaaacatcattataataaaaattagttctccaagtttcttgttgatcaacttgttgtggtggtcctccaattccagcagtagcaatagaagcagaagaattttcagcagtaacaacagaagtagtagcaacaactcctgggtccattccatcagtagtagttcctggaacagaagcagtagtagcagttccagcagctccagcttgtggagcagttctagcctttccttccatgggtcctggtccattagccatagaaatagcagcaatagcatgtggagtcttattattccaaacacacaacttttcaaccttagcttcagtaagataagcaattctaagagtatccttcattctttcaatagccttcttttgagaatcaatatgttgagatcctggaacttcaacttgaaaagtagctccattcttaaaagtaataatagccatttctctctttccagcaagagattcagtataagaaaaaatcttatcattaagagtatgaatttgagtattatgatattcagcacaaagatcagtaatattttgtggagtggaggaagccattgtcccgggcggccgcttctatagtgtcacctaaatcgggtcgaatttgctttcgaatttctgccattcatccgcttattatcacttattcaggcgtacaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 4 - DNA, 5621 bp, synthetic construct
  misc_feature 1..5621: note = ptRNAsup plasmid
  source 1..5621: mol_type = other DNA
tcattagtcttcaatgacgtgtaaaccacggcgctttaagtcctctaacgaatccaacattccccttattaattcaacaggatgcccctcccagtcttcaacaacgccaacaattctcaagggttcgcaggttctataggactgtgttggattaccgggaaattttttgttcgtaagattcggatcgtcttcgaacggtcctgttggctcaactatgtatatgtagccgcgaccctcgaggccagacagtgacatagcaagttcagctccccaaactgctggctccatcaaggctgaaaagtagatgtgcttaagaatacgaccgtcctcgaaatgagagatgaaccctgtggttagcaagtcaccaatcgccaaattggctttggttccatgatagaacggtccttgcacctgcttgtaattatcatgagagatgggaatccaatcttttaccatataacgaacctcctcttgcttattacagcaacgccgcgtctgaattatttctagagaagcatttatcagggttattgtctcatgagcggatacatatttgaatgtatttagaaaaataaacaaataggggttccgcgcacatttccccgaaaagtgcgagtatttacaacgaagacgcaatcactttcttgatcagtgatcgtttcggcgtaattttcagcaaacgatcaaaagtggtgaaaaatatcgttgactcatcgcgccaggtaagtagaatgcaacgcatcgaacggcggcactgattgccagacgataataaaatcaagtgattaactgattgcttgatgaatctttcttcccgagcccggatggtggaatcggtagacacaagggatttaaaatccctcggcgttcgcgctgtgcgggttcaagtcccgctccgggtaccatgggaaagataagaataaaatcaaagcaataagcagtgtcctcggtaccaaattttcgaaaaaagacgctgaaaagcgtcttttttcgttttggtccttacaatcagcagtcagaacttttacgaagaatagtggtcgctcaaccttttctgttgataaaacactctttttgacgtttttacagactaattgaacgtgaagtgtgcaaacgataaaagtgtaggaaaaattgtttgacttataagtctcagaaagtaatatgtgcgcgacgcagcgacgatgagcgataaacaagttcttcgaagcactcgtaagaggcgtgtggtgaggtggccgagaggctgaaggcgctcccctgctaagggagtatgcggtcaaaagctgcatccggggttcgaatccccgcctcaccgccatttgcatctaaaaaaaaaccccgcccctgacagggcggggttttttttttaattatagggataacagggtaatatattcacttccctcacagattcgttcagagataaaagcgttggtaacagttgcctggagtgtgacaaagcgttacacatcgctgtatgcaatgctgaaaatttcagcacttagcgaggtgcgagcaagctggcgcttgcatggtggcgtgcgacaggtataatccacaacgttttcgcgcatacctcttcagtgccgaagtggcgaaatcggtagacgcagttgattcaaaatcaaccgtagaaatacgtgccggttcgagtccggccttcggcaccaaaagtatgtaaatagacctcaactgaggtctttttttatgccaattattgaaggccgctaacgcggcctttttttgtttctggtctccctgcggataaagttgatacccttacctgagttcttctgaaaataacggactaaaaagcagaacgtgcgcgaaaacattaagaaaaattataaaaacccggcataaatggcgagggtttaagcaatcgagcggcagcgtacttaccccgcactcgattagcgggtatactcatgccgcattgtcctcttagttaaatggatataacgagcccctcctaagggctaattgcaggttcgattcctgcaggggacaccatttatcagttcgctcccatccgtaccagtccgcaaaatccaacgcatgagaaagcccccggaagatcaccttccgggggcttttttattgcgctgaaaagcaatccctcgtgaagtaactcaatagtgttctctggtatcgtagtgtgcgtttgtttgccgctatagcgaaataaatcagaaaatcagacgcggtcgttcacttgttcagcaaccagatcaaaagccattgactcagcaagcgttgaccgtataattcacgcgattacacgcgcattgcggtatcaacgcgcccttagctcagttggatagagcaacgaccttctaagtcgtgggccgcaggttcgaatcctgcagggcgcgccattacaattcaatcagttacgccttctttatatcctccataagaaaaaagcccgcacctgacagtgcgggctttttttttcgatcacttctggaagcttgagcacgtgttgacaattaatcatcggcatagtatatcggcatagtataatacgacaaggtgaggaactaaacccctataacccactaagaagaatttatgtcacatctcgcagaactggttgcctcagcgaaggcggccatttcacaggcgtcagatgttgccgcgcttgataatgtgcgcgtcgaatatctaggtaaaaaagggcacctaacccttcagatgacgaccctgcgtgagctgccgccagaagagcgtccggcagctggtgcggttatcaacgaagcgaaagagcaggttcagcaggcgctgaatgcgcgtaaagcggaactggaatccgctgcactgaatgcgcgtctggcggcggaaacgattgatgtctctctgccaggtcgtcgcattgaaaacggcggtctgcatccggttacccgtaccatcgaccgtatcgaatcattcttcggtgagcttggctttaccgtggcaaccgggccggaaatcgaagacgattatcataacttcgatgctctgaacattcctggtcaccacccggcgcgagctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatctcctttaccaacctgaaaggcacgctgcacgacttcctgcgtaacttctttgaggaagatctccagattcgcttccgtccttcctacttcccgtttgccgaaccttctgcagaagtggacgtcatgggtaaaaacggtaaatggctggaagtgctgggctgcgggatggtgcatccgaacgtgcttcgtaacgttggcatcgacccggaagtttactctggtttcggcttcgggatggggatggagcgtctgactatgctacgttacggcgtcaccgacctgcgttcattcttcgaaaacgatctgcgtttcctcaaacagtttaaataacattaccctgttatccctaaataagatttacggattactatcttttcgaaaaaaggcctcccaaatcggggggccttttttattgataacaaaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgactagggataacagggtaat

SEQ ID NO: 5 - DNA, 717 bp, synthetic construct
  misc_feature 1..717: note = chimeric construct CTXVP60 or f238
  source 1..717: mol_type = other DNA
atgctattgctgctatttctatggctaatggaccaggacccatggaaggaaaggctagaactgctccacaagctggagctgctggaactgctactactgcttctgttccaggaactactactgatggaatggacccaggagttgttgctactacttctgttgttactgctgaaaattcttctgcttctattgctactgctggaattggaggaccaccacaacaagttgatcaacaagaaacttggagaactaatttttattataatgatgtttttacttggtctgttgctgatgctccaggatctattctttatactgttcaacattctccacaaaataatccattcactgctgttctttctcaaatgtatgctggatgggctggaggaatgcaattcagattcattgttgctggatctggagtttttggaggaagacttgttgctgctgttattccaccaggaattgaaattggaccaggtcttgaagttagacaatttccacatgttgttattgatgctaggtctcttgaaccagttactattactatgccagaccttagaccaaatatgtatcatccaactggagatccaggacttgttccaactcttgttctttctgtttataataatcttattaatccatttggaggatctacttctgctattcaagttactgttgaaactagaccatctgaagattttgaatttgttatga

SEQ ID NO: 6 - DNA, 1917 bp, synthetic construct
  misc_feature 1..1917: note = human growth hormone plasmid
  source 1..1917: mol_type = other DNA
atggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttag

SEQ ID NO: 7 - DNA, 4814 bp, synthetic construct
  misc_feature 1..4814: note = pUC-hGHR wt-RC55 plasmid
  source 1..4814: mol_type = other DNA
ttatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatatggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttagatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagagaggcacctatctcagcgatctggcgatttcgttcatccatagttgcctgggaccccgtcgtgtagataactacgatacgggagggcttaccatctggccccagtgctgcaatgataccgcgagacccacgctcaccggctccagatttatcagcaataaaccagccagccggaagggcggagcgcagaagtggtcctgcaactttatccgcctccatccagtctatcagttgttgccgggaagctagagtaagtagttcgccagttagtagtttgcgcagcgttgttgccattgctacaggcatcgtggtgtcacgctcgtcgtttggtatggcttcattcagctccggttcccaacgatcaaggcgagttacatgatcccccatgttgtgcagaaaagcggttagctccttcggtcctccgatcgttgtcagaagcaggttggccgcagtgttatcagacatggttatggcagcagagcacagttcgcgtactgtcatgccatccgtaagatgcttttctgtgactggggagtactcaacgaggtcattctgagaatagtgtatgcggcgaccgagttgctcttgcccggcgtcaatacgggacagtaccgcgccacatagcagaactttaaaagtggacatcattggaaaacgttcttcggggcgaaaggactcaaggatcttaccagagttgagatccagttcgatgtaacccactcgtgcacctagctgatcttcagcatcttttactttcaccagcgtttctgggtgagcaaaaacaggaaggcaaaatgccgcaaaaaagggaataagggcgacacggaaatgttgaatggacatcgtagggtaacctgaatattattgaagcatttatcagggttattgtctcatgagcggatacatatttgaatgtatttagaaaaataaacaaataggggttccgcgcacatttccccgaaaagtgccacct

SEQ ID NO: 8 - DNA, 374 bp, synthetic construct
  misc_feature 1..374: note = Hok expression construct
  source 1..374: mol_type = other DNA
cacagctaacaccacgtcgtccctatctgctgccctaggtctatgagtggttgctggataactttacgggcatgcataaggctcgtatgatatattcagggagaccacaacggtttccctctacaaataattttgtttaactttcgggtatcccgattaaggaggtttcgttatgaaattacccaggtcaagtctagtatagtgcgttctgatcgtgtgcttgaccttgctgatcttcacctatctgacgcgtaagtccctgtgtgaaattcgttaccgcgacggccaccgtgaggtcgctgcgtttatggcatacgagagcggtaaataacagaaaaaagcccgcacctgacagtgcgggctttttttttcga

SEQ ID NO: 9 - DNA, 4780 bp, synthetic construct
  misc_feature 1..4780: note = Hok expression construct
  source 1..4780: mol_type = other DNA
atgccatagcatttttatccataagattagcggatcctacctgacgctttttatcgcaactctctactgtttctccatacccgtttttttgggctaacaggaggaattagatctatggatgaattcgaaatgatcaagcgcaatacgtcggagatcatctctgaagaagaattacgtgaggttttaaaaaaagacgaaaaatcggcctatattggctttgaaccatcaggaaaaatccaccttggtcactaccttcagattaagaaaatgattgacctgcaaaatgcaggtttcgacattattatcctgctggccgacctgcatgcgtatcttaatcaaaaaggcgaattggatgagatccgcaagatcggagattataacaagaaagtattcgaggctatgggtctgaaagcaaagtatgtgtatgggtcggagttccagcttgacaaggactatacccttaacgtctaccgtcttgctttgaaaaccaccttaaagcgtgcgcgtcgcagcatggagctgatcgctcgtgaggacgagaacccgaaagtcgctgaggtaatctatcccattatgcaagtcaatgacattcactaccttggggtggatgtcgcagtgggcgggatggaacaacgtaagattcacatgctggcacgcgaactgcttcccaagaaggtcgtgtgcatccacaatccggtattgactggccttgacggggagggaaaaatgtccagctcgaagggaaattttatcgcagttgatgactcccctgaggagattcgtgctaaaatcaagaaggcgtattgtccagcaggcgtagttgagggcaatcccatcatggaaattgcgaaatattttcttgaatacccccttacgattaagcgtccagagaagtttggtggcgatctgacggtcaactcttacgaggagctggaatccttatttaagaataaggaactgcacccaatggaccttaaaaacgcggttgcggaagagttgattaaaatcttagagcccatccgtaagcgtctttaactgcagtttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcattccggcggtagttcagcagggcagaacggcggactctaaatccgcatggcaggggttcaaatcccctccgccggaccaaattcgaaaagcctgctcaacgagcaggcttttttgcatgctcgagcagctcagggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggtacccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcactcggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgct

SEQ ID NO: 10 - DNA, 3198 bp, synthetic construct
  misc_feature 1..3198: note = pUC57-Kan-Kid plasmid
  source 1..3198: mol_type = other DNA
tgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacaccaagcttgcatgcaggcctctgcagtcgacgggcccgggatccgatcacagctaacaccacgtcgtccctatctgctgccctaggtctatgagtggttgctggataactttacgggcatgcataaggctcgtatgatatattcagggagaccacaacggtttccctctacaaataattttgtttaactttataaacacatctaaggaggtaaagacctgtatgttaaaatagcaactaaagaacgaaaatggctggatgcatcgtcgcttggtgcgtcgtaaaagcgatatggaacgtggcgaaatttagctggtctccctggacccgaccgcgggccacgagcagcagggcacccgcccagttttgatcgtgaccccggctgcgttcaaccgtgttacccgtctgccggtcgtggtgccggtgacgagcggtggtaatttcgccagaactgcgggttttgcagtttctctggacggcgttggtattcgtactacgggtgttgtacgctgcgatcaaccgcgtaccatcgacatgaaagctcgtggtggcaagcgccttgagcgcgtgccggaaaccattatgaacgaggttctgggtcgtttaagcaccatcctgacctaacagaaaaaagcccgcacctgacagtgcgggctttttttttcgaatctagatgtattcgcgaggtaccgagctcgaattctctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 11 - DNA, 3132 bp, synthetic construct
  misc_feature 1..3132: note = pUC57-Kan-mazF plasmid
  source 1..3132: mol_type = other DNA
tgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacaccaagcttgcatgcaggcctctgcagtcgacgggcccgggatccgatcacagctaacaccacgtcgtccctatctgctgccctaggtctatgagtggttgctggataactttacgggcatgcataaggctcgtatgatatattcagggagaccacaacggtttccctctacaaataattttgtttaactttactacgacgttactttaaggaggatttttaatggtttcaaggtatgtacccgatatgggagacctgatctgggttgacttcgacccgaccaagggcagcgagcaggcgggtcaccgtccagcagttgtgttgtccccgttcatgtagaataacaagactggtatgtgcctgtgcgttccgtgtaccacgcagagcaaaggctagccgtttgaagtggtgttgtctggtcaagagcgcgatggtgtagccctggcggatcaggttaaaagcattgcatggcgtgcgcgtggcgctaccaagaaaggaaccgtcgctccggaagagctgcaactgatcaaagcgaagatcaacgtgttaattggctaacagaaaaaagcccgcacctgacagtgcgggctttttttttcgaatctagatgtattcgcgaggtaccgagctcgaattctctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 12 - DNA, 2922 bp, synthetic construct
  misc_feature 1..2922: note = pUC57-Kan-timP plasmid
  source 1..2922: mol_type = other DNA
tgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacaccaagcttgcatgcaggcctctgcagtcgacgggcccgggatccgatcacagctaacaccacgtcgtccctatctgctgccctaggtctatgagtggttgctggataactttacgggcatgcataaggctcgtatgatatattcagggagaccacaacggtttccctctacaaataattttgtttaactttataaaataatcaaaataaggaggagacgtcatgaaaataaggtgcttttgtattgtactaatcgtgagcggtgcgctgtagaccgaggttaataacaatcgtagctagtccggcgacaacctgttggttgtgaacaacctgcagtctagcaagtaacagaaaaaagcccgcacctgacagtgcgggctttttttttcgaatctagatgtattcgcgaggtaccgagctcgaattctctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 13 - DNA, 5042 bp, synthetic construct
  misc_feature 1..5042: note = p15A-hGHR wt-RC55 plasmid
  source 1..5042: mol_type = other DNA
ccaattattgaaggccgctaacgcggcctttttttgtttctggtctccctctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaccaccgtcaaaaaaaacggcgctttttagcgccgtttttatttttcaaccttttaggacagcttgacggctacatcattcactttttcttcacaaccggcacggaactcggacggagaggccccggtgcattttttaaatacccgagagaaatagagttgatcgtcaaaaccaacattgcgaccgacggtggcgataggcatccgggtggtggacagaagcagcttcgcctgagagatacgttggtcctcgcgccaggacaggacagaaatcccgagctgctggcggaaaagatgagacagacgagacggagagaggcaaacatgctgtgcgacagaggcgatatcaaaattagagtctgccaggtgatcagagatgtactgacaagcctcgcgtacccgattatccatcggtggatggagggactcgttaatcgcttccatgcgccgcagaagtagttgctcaagcagatttatcgccagcagctcagaatagcgcccttccccttgcccggcgttaatgatttgcccaaacaggtcggagaaatgcggctggtgcgcttcatccgggcgaaagaaccccgtattggcaaatatagacggccagttaagccattcatgccagtaggcgcgcggacgaaagtaaacccactggtgataccattcgcgagcctccggatgacgaccgtagtgatgaatctctcctggcgggaacagaagaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgtaagacccgccaccagatgggcattaaaagagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccatggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttagagtcaaaagcctccgaccggaggcttttgactcctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtccgttgctcaatgtacctataaccagaccgttcagctggatattacggcctttctgaagaccgtaaagaaaaataagcacaagttttatccggcctttattcacattcttgcccgcctgatgaatgctcatccggaatttcgtatggcaatgaaagacggtgagctggtgatatgggattctgttcacccttgttacaccgttttccatgagcaaactgaaacgttttcctctctctggtccgaataccacgacgatttccggcagtttctacacatatattcccaagatgtggcgtgttacggtgaaaacctggcctatttccctaaagggtttattgagaatatgtttttcgtctccgccaatccctgggtgtccttcacctcctttgatctcaacgtggccaatatggacaacttcttcgcccccgttttcaccatgggcaaatattatacgcaaggcgacaaggtgctgatgccgctggcgattcaggttcatcatgccgtttgtgatggcttccatgtcggccgcatgcttaatgaactgcaacagtactgcgatgagtggcagggcggggcgtaa

SEQ ID NO: 14 - protein, 52 aa, synthetic construct
  REGION 1..52: note = Hok protein
MKLPRSSLVWCVLIVCLTLLIFTYLTRKSLCEIRYRDGHREVAAFMAYESGK

SEQ ID NO: 15 - protein, 133 aa, synthetic construct
  REGION 1..133: note = Kid protein
MLKYQLKNENGWMHRRLVRRKSDMERGEIWLVSLDPTAGHEQQGTRPVLIVTPAAFNRVTRLPVVVPVTSGGNFARTAGFAVSLDGVGIRTTGVVRCDQPRTIDMKARGGKRLERVPETIMNEVLGRLSTILT

SEQ ID NO: 16 - protein, 111 aa, synthetic construct
  REGION 1..111: note = mazF protein
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 17 - protein, 41 aa, synthetic construct
  REGION 1..41: note = timP protein
MKIRCFCIVLIVSGALLTEVNNNRSLSGDNLLVVNNLQSSK

SEQ ID NO: 18 - DNA, 717 bp, synthetic construct; identical to SEQ ID NO: 5
  misc_feature 1..717: note = CTX-VP60 chimeric construct
  source 1..717: mol_type = other DNA

SEQ ID NO: 19 - protein, 272 aa, synthetic construct
  REGION 1..272: note = CTX-VP60 chimeric construct
MLLLLFLWLMDQDPWKERLELLHKLELLELLLLLLFQELLLMEWTQELLLLLLLLLLKLLELLLLLLLLELEDHHNKLINKKLGELLLEFLLELLEMMFLLGLLLMLQDLFFLLELFNLLELHKLLELLEHSLLFFLKCMLDGLEECNSDSLLLDLEFLEEDLLLLLFHQELKLDQVLKLDNFHMLLLMLGLLNQLLLLCQTLDQLLECLLELLEQLELLEQDLFQLLFFLFLLELLELLELLLLEHLEDLLLLFKLLLKLDHLKLLELNLL

(19) United States
(12) Patent Application Publication
(10) Pub. No.: US 2026/0366034 A1
(43) Pub. Date: Nov. 19, 2026

(54) Methods for Making and Using Genomically Recoded Cells

(71) Applicant: President and Fellows of Harvard College, Cambridge, MA (US)

(72) Inventors: George M. Church, Brookline, MA (US); Akos Nyerges, Boston, MA (US)

(21) Appl. No.: 19/723,168

(22) Filed: Jun. 29, 2026

Related U.S. Application Data
(63) Continuation of application No. 18/255,160, filed on Dec. 7, 2021, now Pat. No. 12,698,504.
(60) Provisional application No. 63/122,426, filed on Dec. 7, 2020.

(51) Int. Cl. C12N 15/70

(52) U.S. Cl. CPC C12N 15/70

(57) ABSTRACT

Methods making and using genomically recoded cells or organism are provided including genomically recoded cells or organisms that lack the ability to translate a foreign nucleic acid sequence into a polypeptide that may be toxic to the genomically recoded cell or organism.

### CROSS REFERENCE TO RELATED APPLICATIONS

This application is a Continuation Application of U.S. Ser. No. 18/255,160, filed May 31, 2023, which is a National Stage Application under 35 U.S.C. 371 of co-pending PCT application PCT/US2021/062177 designating the United States and filed Dec. 7, 2021; which claims the benefit of U.S. provisional application No. 63/122,426 filed on Dec. 7, 2020 each of which are hereby incorporated by reference in their entireties.

### STATEMENT OF GOVERNMENT INTERESTS

This invention was made with government support under DE-FG02-02ER63445 awarded by the U.S. Department of Energy. The government has certain rights in the invention.

### SEQUENCE LISTING

The instant application contains an updated Sequence Listing which has been submitted electronically in XML format and is hereby incorporated by reference in its entirety. Said XML copy, created on Jul. 9, 2026, is named “Sequence_Listing_010498_01666_ST26” and is 65,536 bytes in size.

### FIELD

The present invention generally relates to methods for making and using genomically recoded cells or organisms.

### BACKGROUND

Industrially synthesized or constructed DNA sequences, regardless of their source or application, are most frequently maintained and manipulated in microbial host cells, e.g., in Escherichia coli (E. coli). Yet a significant fraction of DNA sequences is toxic or pose high metabolic burden to the microbial host cells and thus, unclonable or unstable in microbial host cells. Based on prior studies, 2.6% of all tested microbial genes, approximately 106 genes in an average microbial genome, are toxic in E. coli. A list of 41,821 toxic genes from prokaryotic and eukaryotic genomes is provided in Kimelman A, Levy A, Sberro H, Kidron S, Leavitt A, Amitai G, Yoder-Himes D R, Wurtzel O, Zhu Y, Rubin E M, Sorek R (2012) A vast collection of microbial genes that are toxic to bacteria. Genome Research, 22 (4): 802-809 hereby incorporated by reference in its entirety. DNA constructs, methods of producing proteins and methods to make DNA vaccines are known. See Selas Castiñeiras T, Williams S G, Hitchcock A G, Smith D C (2018) E. coli strain engineering for the production of advanced biopharmaceutical products. FEMS Microbiology Letters, 365 (15); Chakiath C S, Esposito D (2007) Improved recombinational stability of lentiviral expression vectors using reduced-genome Escherichia coli. BioTechniques, 43 (4): 466-470; Pósfai G, Plunkett G, Fehér T, Frisch D, Keil G M, Umenhoffer K, Kolisnychenko V, Stahl B, Sharma S S, De Arruda M (2006) Emergent properties of reduced-genome Escherichia coli. Science, 312 (5776): 1044-1046; Umenhoffer K, Fehér T, Balikó G, Ayaydin F, Pósfai J, Blattner F R, Pósfai G (2010) Reduced evolvability of Escherichia coli MDS42, an IS-less cellular chassis for molecular and synthetic biology applications. Microbial cell factories, 9; Campbell J W, Blattner F R, Plunkett G, Posfai G (2012) Reduced genome E. coli. U.S. Pat. No. 8,178,339 and Cottingham M G, Carroll F, Morris S J, Turner A V, Vaughan A M, Kapulu M C, Colloca S, Siani L, Gilbert S C, Hill A V S (2012) Preventing spontaneous genetic rearrangements in the transgene cassettes of adenovirus vectors. Biotechnology and Bioengineering, 109 (3): 719-728 each of which are hereby incorporated by reference in its entirety.

Furthermore, instability issues frequently prevent vaccine and viral vector construction (Umenhoffer K, Fehér T, Balikó G, Ayaydin F, Pósfai J, Blattner F R, Pósfai G (2010) Reduced evolvability of Escherichia coli MDS42, an IS-less cellular chassis for molecular and synthetic biology applications. Microbial cell factories, 9 and Cottingham M G, Carroll F, Morris S J, Turner A V, Vaughan A M, Kapulu M C, Colloca S, Siani L, Gilbert S C, Hill A V S (2012) Preventing spontaneous genetic rearrangements in the transgene cassettes of adenovirus vectors. Biotechnology and Bioengineering, 109 (3): 719-728) and the rapid analysis of pathogenic viruses, including Flaviviridae, Zika, and SARS-COV-2. Viral reverse genetics has been an indispensable tool to reconstruct viruses purely from sequence data and to aid rapid vaccine development. However, viral genomes are especially cumbersome to clone and to manipulate in bacterial hosts due to instability issue. This instability of eukaryotic or viral sequences is driven by the presence of cryptic bacterial promoters and thus, the expression of toxic proteins or peptides.

Multiple solutions have been proposed to increase stability for difficult-to-clone DNA sequences in microbial host cells. However, these methods suffer from limitations such as high metabolic burden imposed on hosts due to undesired expression from cryptic internal host promoters.

### SUMMARY

Aspects of the present disclosure are directed to method of making genomically recoded cells or organisms. Cells may be genomically recoded as is known in the art by creating unassigned codons. Unassigned codons do not have a cognate tRNA associated with the codon. A codon may be unassigned when the cell is modified to prevent production of the tRNA cognate to the codon. A codon may be unassigned by altering the three nucleotide sequence for the codon. In either example, the cell does not produce a tRNA that recognizes the codon, and so the codon is “unassigned.” Materials and methods useful in the practice of the present disclosure in making and using genomically recoded cells or organisms are provided in WO2017/218727 hereby incorporated by reference in its entirety.

Aspects of the present disclosure are directed to a method for altering a cell, wherein the cell lacks (1) one or more native genes operative to encode one or more tRNA cognate to one or more canonical codons thereby rendering the one or more canonical codons unassigned in the cell or (2) one or more native genes operative to encode one or more release factors. The method provides introducing into the cell a foreign nucleic acid sequence of canonical codons encoding a polypeptide, wherein lack of the cell to produce (1) the one or more tRNA cognate to the one or more canonical codons or (2) the one or more release factors prevents expression of the foreign nucleic acid sequence into the polypeptide.

Aspects of the present disclosure are directed to a method for altering a cell including providing a cell with a foreign nucleic acid sequence of canonical codons encoding a polypeptide, wherein one or more canonical codon sequences within the foreign nucleic acid sequence are altered to produce one or more unassigned codons within the cell, wherein presence of the unassigned codon reduces or prevents expression of the foreign nucleic acid sequence into the polypeptide, wherein the unassigned codon is optionally located upstream of the nucleic acid sequence encoding the protein, wherein the unassigned codon is optionally located in a promoter cognate to the foreign nucleic acid sequence, or wherein the unassigned codon is optionally located within the open reading frame of the foreign nucleic acid sequence.

Aspects of the present disclosure are directed to a method for expressing a foreign nucleic acid sequence to produce a polypeptide in a cell, wherein the cell lacks (1) one or more native genes operative to encode one or more tRNA cognate to one or more canonical codons within the foreign nucleic acid sequence or (2) one or more native genes operative to encode one or more release factors. The method includes providing the cell with the foreign nucleic acid sequence, optionally providing the cell with an expression plasmid or vector that is expressed (1) to provide the one or more tRNA cognate to the one or more canonical codons within the foreign nucleic acid sequence or (2) the one or more release factors, wherein the cell translates the foreign nucleic acid sequence to produce the polypeptide, or optionally, wherein the cell is genetically modified to encode one or more tRNA cognate to one or more canonical codons within the foreign nucleic acid sequence under the influence of an inducible promoter and inducing the inducible promoter to begin translation of the foreign nucleic acid sequence to produce the polypeptide.

In one embodiment, the foreign nucleic acid sequence encodes a protein or peptide or polypeptide or oligopeptide which when expressed is toxic to the cell. In one embodiment, a codon has been eliminated and replaced genome-wide in the genomically recoded organism. The eliminated/replaced codon is termed a forbidden codon or unassigned codon. The forbidden or unassigned codon can be reassigned to incorporate a desired amino acid into a protein or peptide under suitable conditions. In other embodiments, multiple codons can be eliminated and replaced genome-wide in the genomically recoded organism. In one embodiment, tRNAs cognate for the eliminated/replaced forbidden/unassigned codons as well as the associated release factor are eliminated from the genomically recoded organism. In one embodiment, the foreign nucleic acid sequence contains the forbidden/unassigned codon in the open reading frame encoding the protein or peptide. In other embodiments, the foreign nucleic acid sequence contains multiple forbidden/unassigned codons in the open reading frame encoding the protein or peptide. The presence of the forbidden/unassigned codon(s) in the open reading frame encoding the protein or peptide within the foreign nucleic acid sequence prevents translation or proper translation of the protein or peptide. In one embodiment, the improperly translated protein or peptide is degraded in the host cell. In one embodiment, the corresponding cognate tRNAs and associated release factor are provided to the host exogenously to allow proper translation of the protein or peptide. In one embodiment, the corresponding cognate tRNAs and associated release factor are supplied from exogenous expression vectors in an inducible manner which restores translation and the expression for the protein or peptide of interest. Methods according to the present disclosure allows for uncoupling of foreign nucleic acid sequence propagation from its encoded polypeptide expression, which leads to a stable in vivo propagation of the foreign nucleic acid sequence and subsequent high-yield protein production, for example from otherwise toxic genetic elements.

Further features and advantages of certain embodiments of the present disclosure will become more fully apparent in the following description of embodiments and drawings thereof, and from the claims.

### BRIEF DESCRIPTION OF THE DRAWINGS

The patent or application file contains drawings executed in color. Copies of this patent or patent application publication with the color drawings will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 illustrates an example graph of the CTX-VP60 chimeric construct containing many forbidden codons in orf238.

FIG. 2 illustrates an example graph of the sequence of the CTX-VP60 chimeric construct, with UUG and UUA leucine codons marked as “Forbidden Codon” in orf238 in accordance with one or more exemplary embodiments. (SEQ ID Nos: 18-19)

FIG. 3 illustrate a schematic structure of the ptRNAsup plasmid in accordance with an exemplary embodiment.

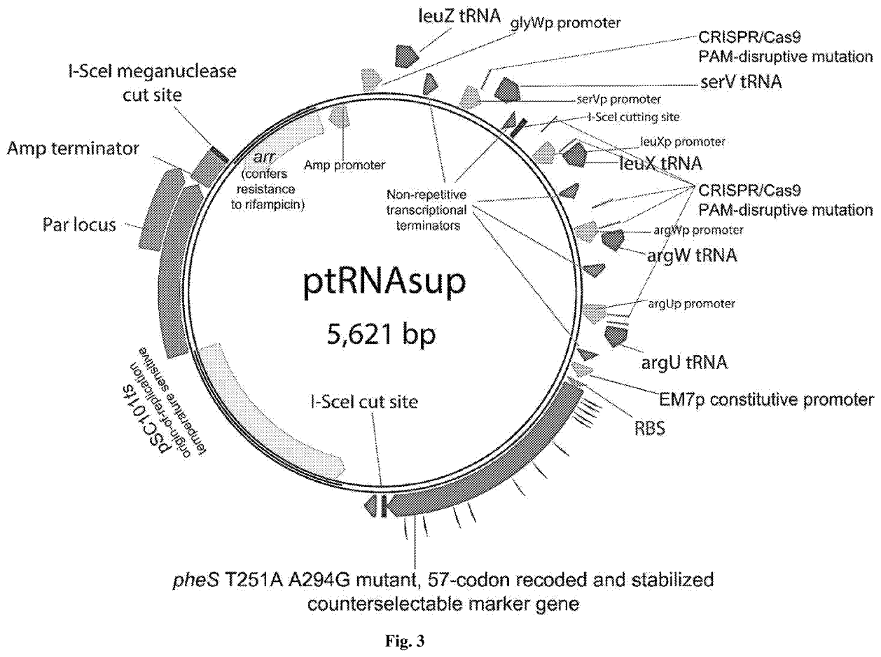

FIG. 4 depicts data from a time-course cell count experiment of the effect of high-level Hok production.

### DETAILED DESCRIPTION

According to certain aspects, the present disclosure provides methods and compositions for maintenance and expression of foreign nucleic acid sequences, such as those encoding polypeptides toxic to the host cell in a genomically recoded organism (GRO). In certain embodiments, in a genomically recoded organism as that term is well understood to those of skill in the art (see Ma N J, Moonan D W, Isaacs F J (2014) Precise manipulation of bacterial chromosomes by conjugative assembly genome engineering. Nature Protocols, 9 (10): 2285-2300 and Lajoie M J, Rovner A J, Goodman D B, Aerni H-R, Haimovich A D, Kuznetsov G, Mercer J A, Wang H H, Carr P A, Mosberg J A, Rohland N, Schultz P G, Jacobson J M, Rinehart J, Church G M, Isaacs F J (2013) Genomically Recoded Organisms Expand Biological Functions. Science, 342 (6156): 357-360 each of which is hereby incorporated by reference in its entirety for making and using genomically recoded organisms), single or multiple canonical genetic codons are eliminated genome-wide and their cognate tRNA(s) and/or release factor (i.e., RF1, prfA) are deleted from the GRO genome. In one embodiment, the foreign nucleic acid sequence contains the eliminated genetic codon(s), such as in the open reading frame of the genetic-elements-of-interest. As a result, an otherwise toxic genetic-element-of-interest can be maintained in the genomically recoded organism. This is because in the absence of the cognate tRNA and/or release factor, the otherwise toxic genetic elements become improperly translated at codons that lack the corresponding tRNA and/or release factor (see Ostrov N, Landon M, Guell M, Kuznetsov G, Teramoto J, Cervantes N, Zhou M, Singh K, Napolitano M G, Moosburner M, Shrock E, Pruitt B W, Conway N, Goodman D B, Gardner C L, Tyree G, Gonzales A, Wanner B L, Norville J E, Lajoie M J, Church G M (2016) Design, synthesis, and testing toward a 57-codon genome. Science, 353 (6301): 819-822; and Ma N J, Isaacs F J (2016) Genomic Recoding Broadly Obstructs the Propagation of Horizontally Transferred Genetic Elements. Cell Systems, 3 (2): 199-207 each of which are hereby incorporated by reference in its entirety. Consequently, cloning and propagating of the foreign nucleic acid sequence in GROs removes the associated fitness cost for genetic-elements-of-interest, for example, where whose (cryptic) expression would be otherwise toxic in organisms that are carrying the canonical genetic code.

According to certain other aspects of the present disclosure, when expression from the genetic-elements-of-interest is desired (e.g., in the production stage of fed-batch fermentation), the cognate tRNAs and/or release factor 1 (e.g., PrfA) are supplied from an exogenous expression vector, for example in an inducible manner, which in turn restores translation and expression for the genetic-elements-of-interest after induction. Therefore, the disclosed methods allow for the stable in vivo propagation and then, high-yield protein production, for example from otherwise toxic or unstable genetic elements.

Vectors according to the present disclosure include those known in the art as being useful in delivering genetic material into a host organism or cell and would include regulators, promoters, nuclear localization signals (NLS), start codons, stop codons, a transgene etc., and any other genetic elements useful for maintenance and expression, as are known to those of skill in the art.

The first reported GRO is of prokaryotic origin, i.e., a bacterium, a strain of E. coli. See Lajoie M J, Rovner A J, Goodman D B, Aerni H-R, Haimovich A D, Kuznetsov G, Mercer J A, Wang H H, Carr P A, Mosberg J A, Rohland N, Schultz P G, Jacobson J M, Rinehart J, Church G M, Isaacs F J (2013) Genomically Recoded Organisms Expand Biological Functions. Science, 342 (6156): 357-360 hereby incorporated by reference in its entirety. Cells of GRO according to the present disclosure include any cell into which foreign nucleic acids can be introduced and expressed as described herein. It is to be understood that the basic concepts of the present disclosure described herein are not limited by cell type for GROs. According to one aspect, the cell is a eukaryotic cell or a prokaryotic cell. According to one aspect, the cell is a yeast cell, bacterial cell, fungal cell, a plant cell or an animal cell. According to one aspect, the cell is a mammalian cell. According to one aspect, the cell is a human cell.

Embodiments of the present disclosure include GROs allowing for the presence of unassigned genetic codons in the genetic code. Exemplary GROs according to the present disclosure include strain E. coli C321.ΔA in which the stop codon UAG is eliminated/replace with UAA genome-wide. The unassigned UAG codon is thus available to be reassigned to encode a desired amino acid. As another example, GROs according to the present disclosure include strain rE.coli-57, in which the AGA, AGG, AGC, AGU, UUA, UUG, and UAG canonical codons in rE.coli-57 are replaced with their respective synonymous codons. See Ostrov N, Landon M, Guell M, Kuznetsov G, Teramoto J, Cervantes N, Zhou M, Singh K, Napolitano M G, Moosburner M, Shrock E, Pruitt B W, Conway N, Goodman D B, Gardner C L, Tyree G, Gonzales A, Wanner B L, Norville J E, Lajoie M J, Church G M (2016) Design, synthesis, and testing toward a 57-codon genome. Science, 353 (6301): 819-822 for the description of rE.coli-57 each of which is hereby incorporated by reference in its entirety. In addition, tRNAs for ΔargU ΔargW ΔserV ΔleuX ΔleuZ and release factor ΔprfA are deleted from the genome of the strain rE.coli-57. In another example, E. coli Syn61 the UCG, UCA, and UAG canonical codons in rE.coli-57 are replaced with their respective synonymous alternatives and ΔserU and ΔserT, and release factor ΔprfA are deleted from the genome. See also Wang K, Fredens J, Brunner S F, Kim S H, Chia T, Chin J W (2016) Defining synonymous codon compression schemes by genome recoding. Nature, 539 (7627): 59-64 for the description of E. coli Syn61. In yet another example, GROs according to the present disclosure include strain Saccharomyces cerevisiae Sc2.0 in which the stop codon UAG is eliminated/replace with UAA genome-wide. See Lajoie M J, Rovner A J, Goodman D B, Aerni H-R, Haimovich A D, Kuznetsov G, Mercer J A, Wang H H, Carr P A, Mosberg J A, Rohland N, Schultz P G, Jacobson J M, Rinehart J, Church G M, Isaacs F J (2013) Genomically Recoded Organisms Expand Biological Functions. Science, 342 (6156): 357-360 and Ostrov N, Landon M, Guell M, Kuznetsov G, Teramoto J, Cervantes N, Zhou M, Singh K, Napolitano M G, Moosburner M, Shrock E, Pruitt B W, Conway N, Goodman D B, Gardner C L, Tyree G, Gonzales A, Wanner B L, Norville J E, Lajoie M J, Church G M (2016) Design, synthesis, and testing toward a 57-codon genome. Science, 353 (6301): 819-822 each of which is hereby incorporated by reference in its entirety.

When a foreign nucleic acid sequence, such as a foreign DNA sequence encoding a polypeptide of interest, containing a forbidden/unassigned codon is introduced into a genomically recoded cell or organism, i.e. which lacks a tRNA for the forbidden/unassigned codon, the foreign DNA sequence is not translated into the polypeptide, as the cell or organism lacks the cellular machinery to do so.

In prokaryotic cells, encountering an unassigned codon during translation leads to ribosomal stalling, and in turn, rescue mechanisms that result in improperly translated proteins with altered C-terminal sequences and/or early termination. In E. coli cells, the tmRNA (ssrA)-SmpB system acts as the primary rescue mechanism leading to the addition of a C-terminal degradation tag (-AANDENYALAA) (SEQ ID NO:1) to peptides at the stalled ribosome. As a consequence, the synthesized protein becomes quickly degraded (see Keiler K C (2015) Mechanisms of ribosome rescue in bacteria. Nature Reviews Microbiology, 13 (5): 285-297 hereby incorporated by reference in its entirety. In eukaryotes, RNA quality control mechanisms quickly eliminate mRNAs that are containing the unassigned codons (see Doma M K, Parker R (2007) RNA Quality Control in Eukaryotes. Cell, 131 (4): 660-668 hereby incorporated by reference in its entirety.)

According to certain aspects of the present disclosure, the maintenance and production of genetic elements, i.e. foreign nucleic acid sequences, where polypeptide or protein expression can be toxic to the cell, can be accomplished in GROs as described herein that where the foreign nucleic acid sequence includes unassigned codons within the cell, i.e. where the cell lacks a cognate tRNA for the unassigned codon.

In some embodiments, a DNA sequence is not part of a cell's natural nucleic acid composition, i.e. is a foreign nucleic acid sequence, and may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources. According to one aspect, a DNA sequence is exogenous/foreign to the cell.

In certain embodiments, genes coding for cognate tRNAs and release factors are deleted from a genomically recoded organism or cell using any method known to those skilled in the art for such deletion. Such deletion creates unassigned codons within the cell as is known to those of skill in the art. In one embodiment, a CRISPR/Cas9 and guide RNA system is used to target the cognate tRNAs and release factors for deletion or editing to render them inoperative. One of skill in the art will readily understand and adapt such methods using readily identifiable literature sources.

The degeneracy of the canonical genetic code allows the same amino acid to be encoded by multiple synonymous codons, as is known in the art. Once a codon is synonymously replaced genome-wide and/or its cognate tRNA and/or associated release factor is eliminated, the genomically recoded organism (GRO) may no longer translate the eliminated codon. The eliminated/replaced codons can be reassigned to desired amino acids. For example in genomically recoded E. coli C321.ΔA, the amber stop codon UAG is replaced genome-wide with the synonymous ochre stop codon UAA. Additional amino acids can be recoded to result in more genomically recoded strains. For example, in rEcoli-57 strain, either one or both of the UUG and UUA Leucine codons can be replaced with the synonymous Leucine codons such as CUU, CUC, CUA or CUG. Codons for Arginine and Serine can be similarly recoded. The replaced codons are also termed forbidden/unassigned codons. Exemplary forbidden/unassigned codons include AGA (Arg), AGG (Arg), AGC (Ser), AGU (Ser), UUG (Leu), UUA (Leu) and UAG (Stop). In addition, the cognate tRNA and/or release factor corresponding to the forbidden/unassigned codons can also be eliminated, i.e. the cell can be altered to prevent expression of the cognate tRNA and/or release factor. Such genomically recoded E. coli can be used to maintain and express DNA sequences that encode for polypeptides or proteins that may be toxic to the cell. For example, when a DNA sequence encodes a protein or peptide that is toxic to the host E. coli stain, it is difficult to propagate the DNA sequence in the host due to toxicity from the expressed protein to the host cells. According to certain aspects, toxicity is determined by a reduction in cell growth rate by at least 1%, 10%, 20%, 30%, 40%, 50%, 60%, 70%, 80%, 90% or 95% compared to growth rate of a corresponding wild type cell. In one embodiment, when the DNA sequence is introduced into the genomically recoded E. coli C321.ΔA strain, if the DNA sequence has the amber stop codon UAG at the end of the open reading frame, the genomically recoded E. coli C321.ΔA would not be able to recognize the stop codon UAG since both the stop codon UAG and/or its cognate tRNA and/or release factor are eliminated in the recoded host E. coli C321.ΔA strain, as is known in the art. The improperly translated protein will be degraded, and as such will not be toxic to the cell. Only when exogenous cognate tRNA for UAG and the associated release factor is provided to the E. coli C321.ΔA stain, will the DNA sequence be properly translated and expressed. In an alternative embodiment, the DNA sequence can have the ochre stop codon UAA at the end of the open reading frame. In such a case, the DNA sequence can be recoded to replace a genetic codon that normally encodes an amino acid in the middle of the open reading frame with the amber stop codon UAG. In this manner, when the recoded DNA sequence is introduced into the genomically recoded E. coli C321.ΔA, the genomically recoded E. coli C321.ΔA would not be able to translate the codon UAG in the middle of the open reading frame since both the codon UAG and its cognate tRNA and/or release factor are eliminated in the recoded host E. coli C321.ΔA strain. Once again, the improperly translated protein will be degraded. When expression of the protein is desired, the codon UAG in the middle of the open reading frame can be reassigned to incorporate the original amino acid in the middle of the open reading frame with exogenously supplied cognate tRNA and release factor to the recoded E. coli C321.ΔA stain. In this manner, the recoded DNA sequence having the codon UAG in the middle of the open reading frame can be properly translated with original amino acid at the UAG codon and expressed. Thus, methods according to the present disclosure allows the maintenance and propagation of the DNA sequence to be separated from its expression so that high-yield protein production can be achieved.

In another embodiment, if the DNA sequence uses e.g. the unassigned codon UUG to encode amino acid Leucine in the middle of the open reading frame, when the DNA sequence is introduced into a genomically recoded E. coli strain having the Leucine codon UUG replaced by its synonymous Leucine codon CUU, the genomically recoded E. coli strain would not be able to translate codon UUG since both the codon UUG and its cognate tRNA and associated release factor are eliminated in the recoded host E. coli strain. The incompletely translated protein will be quickly degraded. Only when exogenous cognate tRNA for UUG recognizing Leucine is provided to the recoded E. coli stain along with the associated release factor, will the DNA sequence be properly translated with Leucine at the UUG codon and expressed. In this manner, the maintenance and propagation of the DNA sequence can be separated from its expression so that high-yield protein production can be achieved.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables and accompanying claims.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art. Other equivalent embodiments will be apparent in view of the present disclosure, figures and accompanying claims.

### Example I

### Maintenance of Toxic DNA Sequence in a Genomically Recoded Organism

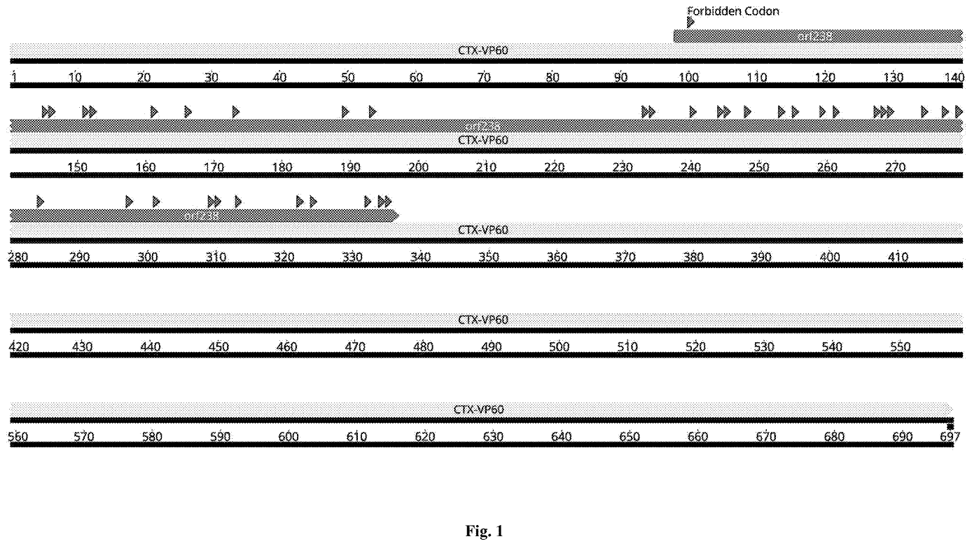

In this example, a chimeric gene CTX-VP60 was used. The chimeric gene CTX-VP60 is composed of a synthetic gene of the structural capsid protein VP60 of rabbit hemorrhagic disease (RHD) virus fused to the B subunit of cholera toxin (CTX). This chimeric gene is extremely unstable in industrial E. coli strains, due to severe toxicity See Pósfai G, Plunkett G, Fehér T, Frisch D, Keil G M, Umenhoffer K, Kolisnychenko V, Stahl B, Sharma S S, De Arruda M (2006) Emergent properties of reduced-genome Escherichia coli. Science, 312 (5776): 1044-1046 and Umenhoffer K, Fehér T, Balikó G, Ayaydin F, Pósfai J, Blattner F R, Pósfai G (2010) Reduced evolvability of Escherichia coli MDS42, an IS-less cellular chassis for molecular and synthetic biology applications. Microbial cell factories, 9 each of which are hereby incorporated by reference in its entirety for the description of CTX-VP60. The severe toxicity of CTX-VP60 is caused by an internal, leucine-rich protein-coding gene, termed orf238. In rE.coli-57 ΔleuX ΔleuZ, the plasmid including the chimeric gene sequence encoding CTX-VP60 can be maintained in the recoded rE.coli-57 ΔleuX ΔleuZ, as compared to wild type E. coli. Since orf238 in the chimeric gene CTX-VP60 includes a large number of “forbidden” leucine codons (i.e., UUG and UUA) in its open reading frame (see FIG. 1), proper translation of CTX-VP60 is not possible in the recoded rE.coli-57 ΔleuX ΔleuZ. Further, the LeuX and LeuZ tRNAs responsible for the recognition of mRNA codons UUG and UUA are eliminated from the recoded strain making translation not possible.

### Example II

### Expression of Toxic Protein from a DNA Sequence in a Genomically Recoded Organism

In an exemplary embodiment, expression of toxic protein from a DNA sequence can be achieved in a genomically recoded organism. In this example, a counterselectable expression plasmid (ptRNAsup, and derivatives) that, after delivery into the target expression host lacking cognate tRNA genes and/or Release Factor 1, provides expression for necessary tRNAs for the production of the toxic target protein(s). Counterselection markers are generally described in Miyazaki K (2015) Molecular engineering of a PheS counterselection marker for improved operating efficiency in Escherichia coli. BioTechniques, 58 (2): 86-88 hereby incorporated by reference in its entirety.

In an alternative application, ptRNAsup and derivatives can allow for the CRISPR/Cas9-mediated deletion of endogenous tRNA and release factor genes by supplementing the host-cell with tRNA genes and/or Release Factor during the genome editing process. Then, once the chromosomal copies of these tRNA genes and/or Release Factor(s) are deleted, the ptRNAsup plasmid is selectively eliminable from the host at an elevated temperature and/or by simultaneous counterselection.

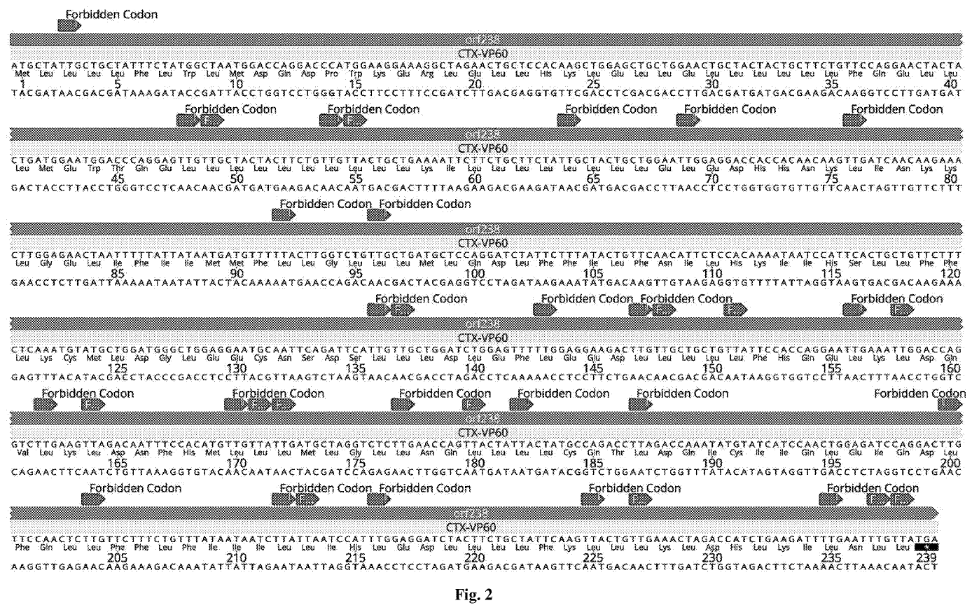

The ptRNAsup plasmid includes the following features for selective elimination.
Temperature-sensitive origin-of-replication: The pSC101ts origin of replication allows stable low copy number maintenance and tRNA and/or RF1 expression at 30-34 degrees Celsius but eliminated at elevated temperatures (i.e., >41 degrees Celsius).
Constitutively expressed arr (antibiotic resistance) gene: Provides resistance for the host to rifampicin (Rifampin) at 100-300 ug/mL concentration and thus allows selection for the presence of the ptRNAsup plasmid. Other suitable resistance genes (incl. cat, bla, aph (9)-Ia (Spec-R), Aac3-Ia (Gent-R)) can be used instead of arr, and these genes are widely known to those of skill in the art.
Constitutively expressed pheS T251A+A294G mutant gene: Derivative of the native E. coli MG1655 pheS gene, provides stringent counterselection against ptRNAsup in the presence of 4-chloro-phenylalanine (4CP) [27, 28] in E. coli and related Enterobacteria. Other counterselectable markers that can confer similar functions include ccdB, rpsL, tetA, pyrF, sacB, and others that are widely known to those of skill in the art.
Constitutively or inducibly expressed tRNA genes and/or prfA (Release Factor 1) gene. The ptRNAsup plasmid variant that is displayed on FIG. 2 expresses leuZ, serV, leuX, argW, and argU from their native constitutive promoter. Inducible expression systems that can substitute the native promoter of these genes are available and widely known for people who are skilled in the art. The native constitutive promoters of these genes (leuZ, serV, leuX, argW, and argU) have been modified on a way to disrupt spCas9's PAM (NRG) site thereby allowing for the genomic deletion of the wild-type leuZ, serV, leuX, argW, and argU by Cas9-mediated recombineering or other alternative genome editing technologies.
I-SceI meganuclease cutting sites: Allows the elimination (by cutting) the ptRNAsup plasmid by the inducible expression of the I-SceI meganuclease. I-SceI recognizes and cleaves within an 18-base pair sequence 5′-TAGGGATAACAGGGTAAT (SEQ ID NO:2) that is not represented on bacterial genomes, and therefore it can be specifically directed to cleave ptRNAsup.
In alternative version of ptRNAsup, the tRNA genes can be replaced or extended with any inducible or constitutive promoter driven tRNA gene(s) and or Release Factor gene, i.e., prfA.

### Example III

### Expression of Toxic Hok Protein from a DNA Sequence in a Genomically Recoded Organism

The bacterial Hok protein (SEQ ID NO:14) is the toxic component of a type I toxin-antitoxin (TA) system. When overexpressed, it kills cells within minutes by causing the collapse of the transmembrane potential and arrest of respiration (see Pedersen et al., Multiple hok genes on the chromosome of Escherichia coli, Molecular Microbiology, vol. 32, issue 5, pages 1090-1102 (1999; PubMed: 10361310). Due to its toxicity to bacterial cells, including E. coli, the high-level production of the Hok protein is exceptionally challenging.

According to one aspect, methods are provided to maintain constitutively expressed Hok production plasmids in E. coli cells and produce the functional Hok protein by controlling the availability of an aminoacylated tRNA that suppresses an unassigned codon inside the Hok gene. A Hok expression construct was constructed that contained a TAG unassigned codon at amino acid position 10 (SEQ ID NO: 8 ProC-maxRBS-hok) and cloned into a high-copy-number pUC-derived pJET1.2 plasmid. In wild-type E. coli cells, the TAG codon at this position is recognized by the cell's endogenous Release Factor 1 (PrfA) and thus terminates the translation of the otherwise toxic Hok protein. According to one aspect, the TAG-containing Hok expression construct (SEQ ID NO:8 ProC-maxRBS-hok) is stably maintained in common E. coli expression hosts, including E. coli DH10b and MDS42 cells. The pUC-ProC-maxRBS-hok construct was transformed into C321.ΔA.exp (Addgene strain #49018, Lajoie M J, Rovner A J, Goodman D B, Aerni H-R, Haimovich A D, Kuznetsov G, Mercer J A, Wang H H, Carr P A, Mosberg J A, Rohland N, Schultz P G, Jacobson J M, Rinehart J, Church G M, Isaacs F J (2013) Genomically Recoded Organisms Expand Biological Functions. Science, 342 (6156): 357-360. https://doi.org/10.1126/science.1241459). In C321.ΔA.exp, the RF1 is deleted from the genome, and therefore the TAG codon is unassigned. C321.ΔA.exp cells displayed normal growth in the presence of pUC-ProC-maxRBS-hok. The same pUC-ProC-maxRBS-hok construct was transformed into C321.ΔA.exp cells containing an inducible Methanocaldococcus jannaschii TyrRS tyrosine-tRNA ligase and the corresponding Tyr tRNA (SEQ ID NO:9 pEVOL_MJWTyRS-CUA). When induced with 0.2% arabinose in the growth medium, this pEVOL_MJWTyRS-CUA plasmid produces tyrosine-charged tRNAs with CUA anticodon, thus suppressing unassigned TAG codons in the C321.ΔA.exp cells by incorporating tyrosine at TAG codons into polypeptides during ribosomal protein synthesis.

These C321.ΔA.exp+pEVOL_MJWTyRS-CUA+pUC-ProC-maxRBS-hok cells displayed normal growth rate without the inducer and in the presence of 1% glucose that further represses the induction of pEVOL_MJWTyRS-CUA. In the presence of 0.2% arabinose in the growth medium, however, the cells displayed no growth even after 24 hours of incubation. Similarly, restreaking >10\hat{ }9 bacterial cells to agar plates containing 0.2% arabinose resulted in no growth after 48 hours at 37 degrees Celsius. Control C321.ΔA.exp+pEVOL_MJWTyRS-CUA and C321.ΔA.exp+pUC-ProC-maxRBS-hok cells grew normally in the presence of arabinose inducer.

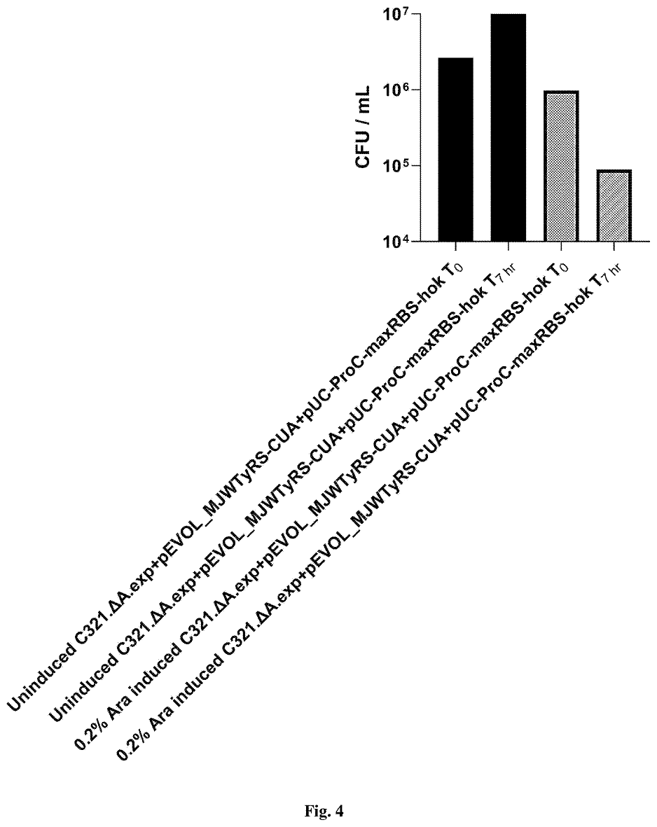

The effect of high-level Hok production was characterized by a time-course cell count experiment (see FIG. 4). In this experiment, an overnight starter culture of C321.ΔA.exp+pEVOL_MJWTyRS-CUA+pUC-ProC-maxRBS-hok cells from 1% glucose-containing medium was inoculated into both selective medium without inducer and separately, into selective medium with 0.2% arabinose inducer and incubated both aerobically at 37 degrees Celsius in a shaking incubator. Next, the viable cell count was assayed under both conditions at the time point of inoculation and after 7 hours of growth by plating cells on nonselective agar plates with 1% glucose. While the uninduced culture grew rapidly, approximately 90% of the arabinose-induced cells died after 7 hours of induction, demonstrating the extreme toxicity of functional Hok production.

### Example IV

### Additional Toxic Proteins as Targets

The protocol of Example III was carried out to test the expression of additional proteins in the C321.ΔA.exp+pEVOL_MJWTyRS-CUA expression system based on the unassigned TAG codon. The Kid (SEQ ID NO:15), mazF (SEQ ID NO:16), and timP (SEQ ID NO:17) toxic proteins were selected as targets. The Kid protein is the toxin part encoded by the parD operon of plasmid R1 (See Kimelman A, Levy A, Sberro H, Kidron S, Leavitt A, Amitai G, Yoder-Himes D R, Wurtzel O, Zhu Y, Rubin E M, Sorek R (2012) A vast collection of microbial genes that are toxic to bacteria. Genome Research, 22 (4): 802-809 and Hargreaves D, Santos-Sierra S, Giraldo R, Sabariegos-Jareño R, Cueva-Méndez G de la, Boelens R, Díaz-Orejas R, Rafferty J B (2002) Structural and Functional Analysis of the Kid Toxin Protein from E. coli Plasmid R1. Structure, 10 (10): 1425-1433). MazF is an mRNA interferase protein, which, upon expression, cleaves bacterial mRNAs in a sequence-specific manner, resulting in cellular growth arrest and cell death (see Kamada K, Hanaoka F, Burley S K (2003) Crystal Structure of the MazE/MazF Complex: Molecular Bases of Antidote-Toxin Recognition. Molecular Cell, 11 (4): 875-884 and Simanshu D K, Yamaguchi Y, Park J-H, Inouye M, Patel D J (2013) Structural Basis of mRNA Recognition and Cleavage by Toxin MazF and Its Regulation by Antitoxin MazE in Bacillus subtilis. Molecular Cell, 52 (3): 447-458.) The timP protein is a small, recently discovered bacterial toxin that results0 in rapid cell death upon induction (see Andresen L, Martínez-Burgo Y, Zangelin J N, Rizvanovic A, Holmqvist E (2020) The Small Toxic Salmonella Protein TimP Targets the Cytoplasmic Membrane and Is Repressed by the Small RNA TimR. mBio, 11 (6) https://doi.org/10.1128/mBio.01659-20).

High-copy-number expression plasmids containing a strong constitutive bacterial promoter and strong ribosome binding site in front of the Kid, mazF, and timP genes containing two instances of the unassigned TAG codon were constructed. These plasmids are SEQ ID NO:10 pUC57-Kan-Kid, SEQ ID NO: 11 pUC57-Kan-mazF, and SEQ ID NO:12 pUC57-Kan-timP, respectively.

Next, the pUC57-Kan-Kid, pUC57-Kan-mazF, and pUC57-Kan-timP were transformed into C321.ΔA.exp cells containing an inducible Methanocaldococcus jannaschii TyrRS tyrosine-tRNA ligase and the corresponding Tyr tRNA (SEQ ID NO:9 pEVOL_MJWTyRS-CUA). When induced with 0.6% arabinose in the growth medium, this pEVOL_MJWTyRS-CUA plasmid produces tyrosine-charged tRNAs with CUA anticodon, thus suppressing unassigned TAG codons in the C321.ΔA.exp cells.

These C321.ΔA.exp+pEVOL_MJWTyRS-CUA+pUC57-Kan-Kid or +pUC57-Kan-mazF, or +pUC57-Kan-timP containing cells displayed only slightly reduced growth rate without the inducer and in the presence of 1% glucose that further represses the induction of pEVOL_MJWTyRS-CUA. In the presence of 0.6% arabinose inducer in the growth medium, the cells displayed no or minimal growth due to the high-level production of the toxic protein. The growth was measured as the optical density of the culture (OD₆₀₀), after 24 hours of incubation at 32 degrees Celsius (see Table 1). These experiments demonstrate the tight regulation of toxic protein expression based on the inducible supply of charged tRNAs suppressing an unassigned codon in genomically recoded organisms.

Table 1 below includes data of the maintenance of constitutively expressed, toxic Kid, mazF, and timP expression constructs in E. coli C321.ΔA cells and these proteins' inducible expression and subsequent toxicity by suppressing the unassigned TAG codon.

| Strain | Induction | Replicate | OD₆₀₀ |
|---|---|---|---|
| C321.ΔA.exp + pEVOL_MJWTyRS-CUA + pUC57-Kan-Kid | Uninduced | 1 | 1.66 |
| C321.ΔA.exp + pEVOL_MJWTyRS-CUA + pUC57-Kan-Kid | Uninduced | 2 | 1.63 |
| C321.ΔA.exp + pEVOL_MJWTyRS-CUA + pUC57-Kan-Kid | Uninduced | 3 | 1.81 |
| C321.ΔA.exp + pEVOL_MJWTyRS-CUA + pUC57-Kan-mazF | Uninduced | 1 | 1.98 |
| C321.ΔA.exp + pEVOL_MJWTyRS-CUA + pUC57-Kan-mazF | Uninduced | 2 | 1.82 |
| C321.ΔA.exp + pEVOL_MJWTyRS-CUA + pUC57-Kan-mazF | Uninduced | 3 | 1.69 |
| C321.ΔA.exp + pEVOL_MJWTyRS-CUA + pUC57-Kan-timP | Uninduced | 1 | 1.62 |
| C321.ΔA.exp + pEVOL_MJWTyRS-CUA + pUC57-Kan-timP | Uninduced | 2 | 1.63 |
| C321.ΔA.exp + pEVOL_MJWTyRS-CUA + pUC57-Kan-timP | Uninduced | 3 | 1.58 |
| C321.ΔA.exp + pEVOL_MJWTyRS-CUA + pUC57-Kan-Kid | Induced with Arabinose | 1 | 0 |
| C321.ΔA.exp + pEVOL_MJWTyRS-CUA + pUC57-Kan-Kid | Induced with Arabinose | 2 | 0 |
| C321.ΔA.exp + pEVOL_MJWTyRS-CUA + pUC57-Kan-Kid | Induced with Arabinose | 3 | 0.62 |
| C321.ΔA.exp + pEVOL_MJWTyRS-CUA + pUC57-Kan-mazF | Induced with Arabinose | 1 | 0.01 |
| C321.ΔA.exp + pEVOL_MJWTyRS-CUA + pUC57-Kan-mazF | Induced with Arabinose | 2 | 0.05 |
| C321.ΔA.exp + pEVOL_MJWTyRS-CUA + pUC57-Kan-mazF | Induced with Arabinose | 3 | 0 |
| C321.ΔA.exp + pEVOL_MJWTyRS-CUA + pUC57-Kan-timP | Induced with Arabinose | 1 | 0 |
| C321.ΔA.exp + pEVOL_MJWTyRS-CUA + pUC57-Kan-timP | Induced with Arabinose | 2 | 0 |
| C321.ΔA.exp + pEVOL_MJWTyRS-CUA + pUC57-Kan-timP | Induced with Arabinose | 3 | 0 |

### Example V

### Human Growth Hormone Receptor as a Toxic Gene

The gene encoding the human growth hormone receptor (hGHR, SEQ ID NO:6 hGHR_wt) displays a high level of instability and is extremely toxic to E. coli bacterial expression hosts. See Bieth E, Cahoreau C, Cholin S, Molinas C, Cerutti M, Rochiccioli P, Devauchelle G, Tauber M (1997) Human growth hormone receptor: cloning and expression of the full-length complementary DNA after site-directed inactivation of a cryptic bacterial promoter. Gene, 194 (1): 97-105. Instability is believed to result from a cryptic bacterial promoter inside the wild-type sequence of hGHR that provides a high level of expression for an unknown downstream protein sequence.

The toxicity of SEQ ID NO:6 hGHR_wt was characterized by cloning the sequence into a high-copy-number, pUC-57 derived plasmid. The cloning of hGHR_wt was carried out in an E. coli host (Lucigen CopyCutter™ EPI400™ cells) that reduces the copy number of the pUC-hGHR_wt plasmid to medium-copy-number, thus reducing the expression level of the toxic protein by an order of magnitude (see Haskins, D. (2004) Epicentre Forum 11 (5), 6.).

The pUC-hGHR_wt plasmid was transformed into various E. coli cloning and industrial strains, including E. coli TOP10 and MDS42. The high toxicity of pUC-hGHR_wt to E. coli cells, is described in Bieth E, Cahoreau C, Cholin S, Molinas C, Cerutti M, Rochiccioli P, Devauchelle G, Tauber M (1997) Human growth hormone receptor: cloning and expression of the full-length complementary DNA after site-directed inactivation of a cryptic bacterial promoter. Gene, 194 (1): 97-105 prevents obtaining transformants containing intact copies of pUC-hGHR_wt.

Recoded plasmid-borne versions of hGHR_wt, termed pUC-hGHR_wt-RC55 and p15A-hGHR_wt-RC55 containing the wild-type non-recoded hGHR sequence and a recoded antibiotic resistance marker, together with a high-copy-number pUC or a medium copy-number p15A bacterial origin-of-plasmid-replication, respectively were synthesized. These sequences are provided as SEQ ID NO:7 pUC-hGHR_wt-RC55 and SEQ ID NO:13 p15A-hGHR_wt-RC55.

Next, pUC-hGHR_wt-RC55 and p15A-hGHR_wt-RC55 were transformed into E. coli Syn61 and E. coli Syn61A3 (ev5). The recoded E. coli Syn6143 (ev5) lacks the serU and serT tRNA genes that encode serine tRNAs with CGA and UGA anticodons and recognize serine codons TCG and TCA. Furthermore, E. coli Syn6143 (ev5) also lacks the gene encoding Release Factor 1, prfA. Consequently, in E. coli Syn6143 (ev5), codons TCG, TCA, and TAG are unassigned and coding sequences having these codons are not expressed in this host. E. coli Syn61 and Syn6143 (ev5) are described in Fredens J, Wang K, Torre D de la, Funke L F H, Robertson W E, Christova Y, Chia T, Schmied W H, Dunkelmann D L, Beránek V, Uttamapinant C, Llamazares A G, Elliott T S, Chin J W (2019) Total synthesis of Escherichia coli with a recoded genome. Nature,:1. https://doi.org/10.1038/s41586-019-1192-5 and Robertson W E, Funke L F H, Torre D de la, Fredens J, Elliott T S, Spinck M, Christova Y, Cervettini D, Böge F L, Liu K C, Buse S, Maslen S, Salmond G P C, Chin J W (2021) Sense codon reassignment enables viral resistance and encoded polymer synthesis. Science, 372 (6546): 1057-1062. The whole genome sequence of Syn6143 (ev5) is available at NCBI Genbank as CP071799.1.

Colonies of Syn6143 (ev5) transformed with pUC-hGHR_wt-RC55 and p15A-hGHR_wt-RC55 display normal growth rate on selective medium. Syn61 transformed with p15A-hGHR_wt-RC55 displays abnormal, slow growth. Repeated transformation of pUC-hGHR_wt-RC55 into Syn61 yields no colonies containing the intact plasmid.

### Example VI

### Embodiments

The present disclosure provides a method for altering a cell, wherein the cell lacks expression of one or more tRNA cognate to one or more canonical codons or lacks expression of one or more release factors or otherwise lacks (1) one or more native genes operative to encode one or more tRNA cognate to one or more canonical codons thereby rendering the one or more canonical codons unassigned in the cell or (2) one or more native genes operative to encode one or more release factors The method includes introducing into the cell a foreign nucleic acid sequence of canonical codons encoding a polypeptide, wherein lack of the cell to produce (1) the one or more tRNA cognate to the one or more canonical codons or (2) the one or more release factors prevents expression of the foreign nucleic acid sequence into the polypeptide. According to one aspect, the cell is a eukaryotic cell. According to one aspect, the cell is a microbial cell. According to one aspect, the cell is a bacterial cell. According to one aspect, the cell is a yeast cell, a plant cell or a mammalian cell. According to one aspect, the cell is a human cell. According to one aspect, the polypeptide is a protein. According to one aspect, the polypeptide is toxic to the cell. According to one aspect, the polypeptide is toxic to the cell, wherein toxicity is determined by a reduction in cell growth rate by at least 1%, 10%, 50%, or 90% compared to growth rate of a corresponding wild type cell. According to one aspect, the polypeptide is a protein that is toxic to the cell. According to one aspect, the polypeptide is a protein that is toxic to the cell and wherein lack of the cell to produce (1) the one or more tRNA cognate to the one or more canonical codons or (2) the one or more release factors prevents expression of the foreign nucleic acid sequence into the polypeptide, thereby reducing toxicity to the cell of the polypeptide compared to a non-recoded cell of the same strain. According to one aspect, the cell is grown to produce a population of cells including the foreign nucleic acid sequence. According to one aspect, one or more tRNA cognate to the one or more unassigned codons are provided within the cell, and wherein the cell expresses the foreign nucleic acid sequence into the polypeptide.

The present disclosure provides a method for altering a cell including providing a cell with a foreign nucleic acid sequence of canonical codons encoding a polypeptide, wherein one or more canonical codon sequences within the foreign nucleic acid sequence are altered to produce one or more unassigned codons within the cell, wherein presence of the unassigned codon reduces or prevents expression of the foreign nucleic acid sequence into the polypeptide, wherein the unassigned codon is optionally located upstream of the nucleic acid sequence encoding the protein, wherein the unassigned codon is optionally located in a promoter cognate to the foreign nucleic acid sequence, or wherein the unassigned codon is optionally located within the open reading frame of the foreign nucleic acid sequence. According to one aspect, the cell is a eukaryotic cell. According to one aspect, the cell is a microbial cell. According to one aspect, the cell is a bacterial cell. According to one aspect, the cell is a yeast cell, a plant cell or a mammalian cell. According to one aspect, the cell is a human cell. According to one aspect, the polypeptide is a protein. According to one aspect, the polypeptide is toxic to the cell. According to one aspect, the polypeptide is toxic to the cell, wherein toxicity is determined by a reduction in cell growth rate by at least 1%, 10%, 50%, or 90% compared to growth rate of a corresponding wild type cell. According to one aspect, the polypeptide is a protein that is toxic to the cell. According to one aspect, the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain. According to one aspect, one or more tRNA cognate to the one or more unassigned codons are provided within the cell, and wherein the cell expresses the foreign nucleic acid sequence into the polypeptide. According to one aspect, the microbial cell is grown to produce a population of microbial cells including the foreign nucleic acid sequence.

The present disclosure provides a method for expressing a foreign nucleic acid sequence to produce a polypeptide in a cell, wherein the cell lacks expression of one or more tRNA cognate to one or more canonical codons or lacks expression of one or more release factors or otherwise lacks (1) one or more native genes operative to encode one or more tRNA cognate to one or more canonical codons within the foreign nucleic acid sequence or (2) one or more native genes operative to encode one or more release factors The method includes providing the cell with the foreign nucleic acid sequence, optionally providing the cell with an expression plasmid or vector that is expressed (1) to provide the one or more tRNA cognate to the one or more canonical codons within the foreign nucleic acid sequence or (2) the one or more release factors, wherein the cell translates the foreign nucleic acid sequence to produce the polypeptide, or optionally, wherein the cell is genetically modified to encode one or more tRNA cognate to one or more canonical codons within the foreign nucleic acid sequence under the influence of an inducible promoter and inducing the inducible promoter to begin translation of the foreign nucleic acid sequence to produce the polypeptide. According to one aspect, the (1) one or more genes encoding one or more tRNA cognate to one or more canonical codons within the foreign nucleic acid sequence or (2) one or more genes encoding one or more release factors are under the influence of a constitutive promoter. According to one aspect, the (1) one or more genes encoding one or more tRNA cognate to one or more canonical codons within the foreign nucleic acid sequence or (2) one or more genes encoding one or more release factors are under the influence of an inducible promoter. According to one aspect, the expression plasmid expresses (1) the one or more tRNA cognate to the one or more canonical codons within the foreign nucleic acid sequence or (2) the one or more release factors at a first temperature but does not express (1) the one or more tRNA cognate to the one or more canonical codons within the foreign nucleic acid sequence or (2) the one or more release factors at a second temperature that is higher than the first temperature. According to one aspect, the expression plasmid includes a resistance gene allowing selection of cells including the expression plasmid. According to one aspect, the expression plasmid includes a counterselectable gene allowing counterselection of cells including the expression plasmid. According to one aspect, the expression plasmid includes a nuclease cutting site. According to one aspect, (1) one or more native genes encoding one or more tRNA cognate to one or more canonical codons within the foreign nucleic acid sequence or (2) one or more native genes encoding one or more release factors are rendered inoperative by CRISPR-Cas9 mediated genome editing or removal of the (1) one or more native genes encoding one or more tRNA cognate to one or more canonical codons within the foreign nucleic acid sequence or (2) one or more native genes encoding one or more release factors. According to one aspect, the cell is grown to produce a population of cells including the foreign nucleic acid sequence. According to one aspect, the cell is a eukaryotic cell. According to one aspect, the cell is a microbial cell. According to one aspect, the cell is a bacterial cell. According to one aspect, the cell is a yeast cell, a plant cell or a mammalian cell. According to one aspect, the cell is a human cell. According to one aspect, the polypeptide is a protein. According to one aspect, the polypeptide is toxic to the cell. According to one aspect, the polypeptide is toxic to the cell, wherein toxicity is determined by a reduction in cell growth rate by at least 1%, 10%, 50%, or 90% compared to growth rate of a corresponding wild type cell. According to one aspect, the polypeptide is a protein that is toxic to the cell. According to one aspect, the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

The present disclosure provides an altered cell lacking expression of one or more tRNA cognate to one or more canonical codons or lacking expression of one or more release factors or otherwise lacking (1) one or more native genes operative to encode one or more tRNA cognate to one or more canonical codons or (2) one or more native genes operative to encode one or more release factors and including a foreign nucleic acid sequence of canonical codons encoding a polypeptide. According to one aspect, the cell is a eukaryotic cell. According to one aspect, the cell is a microbial cell. According to one aspect, the cell is a bacterial cell. According to one aspect, the cell is a yeast cell, a plant cell or a mammalian cell. According to one aspect, the cell is a human cell. According to one aspect, the polypeptide is a protein. According to one aspect, the polypeptide is toxic to the cell. According to one aspect, the polypeptide is toxic to the cell, wherein toxicity is determined by a reduction in cell growth rate by at least 1%, 10%, 50%, or 90% compared to growth rate of a corresponding wild type cell. According to one aspect, the polypeptide is a protein that is toxic to the cell. According to one aspect, the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

The present disclosure provides an altered cell including a foreign nucleic acid sequence of canonical codons encoding a polypeptide, wherein the foreign nucleic acid sequence is recoded to include one or more unassigned codons replacing one or more target canonical codons, wherein the unassigned codon is optionally located upstream of the foreign nucleic acid sequence encoding the protein, wherein the unassigned codon is optionally located in a promoter cognate to the foreign nucleic acid sequence, or wherein the unassigned codon is optionally located within the open reading frame of the foreign nucleic acid sequence. According to one aspect, the cell is a eukaryotic cell. According to one aspect, the cell is a microbial cell. According to one aspect, the cell is a bacterial cell. According to one aspect, the cell is a yeast cell, a plant cell or a mammalian cell. According to one aspect, the cell is a human cell. According to one aspect, the polypeptide is a protein. According to one aspect, the polypeptide is toxic to the cell. According to one aspect, the polypeptide is toxic to the cell, wherein toxicity is determined by a reduction in cell growth rate by at least 1%, 10%, 50%, or 90% compared to growth rate of a corresponding wild type cell. According to one aspect, the polypeptide is a protein that is toxic to the cell. According to one aspect, the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

The present disclosure provides an altered cell lacking expression of one or more tRNA cognate to one or more canonical codons or lacking expression of one or more release factors or otherwise lacking (1) one or more native genes operative to encode one or more tRNA cognate to one or more canonical codons within a foreign nucleic acid sequence encoding a polypeptide or (2) one or more native genes operative to encode one or more release factors and including the foreign nucleic acid sequence, an expression plasmid or vector encoding the one or more tRNA cognate to the one or more canonical codons within the foreign nucleic acid sequence or (2) the one or more release factors. According to one aspect, the cell is a eukaryotic cell. According to one aspect, the cell is a microbial cell. According to one aspect, the cell is a bacterial cell. According to one aspect, the cell is a yeast cell, a plant cell or a mammalian cell. According to one aspect, the cell is a human cell. According to one aspect, the polypeptide is a protein. According to one aspect, the polypeptide is toxic to the cell. According to one aspect, the polypeptide is toxic to the cell, wherein toxicity is determined by a reduction in cell growth rate by at least 1%, 10%, 50%, or 90% compared to growth rate of a corresponding wild type cell. According to one aspect, the polypeptide is a protein that is toxic to the cell. According to one aspect, the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

1. A method for altering a cell, wherein the cell lacks expression of one or more tRNA cognate to one or more canonical codons or lacks expression of one or more release factors or otherwise lacks (1) one or more native genes operative to encode one or more tRNA cognate to one or more canonical codons thereby rendering the one or more canonical codons unassigned in the cell or (2) one or more native genes operative to encode one or more release factors comprising
introducing into the cell a foreign nucleic acid sequence of canonical codons encoding a polypeptide, wherein lack of the cell to produce (1) the one or more tRNA cognate to the one or more canonical codons or (2) the one or more release factors prevents expression of the foreign nucleic acid sequence into the polypeptide.

2. The method of claim 1 wherein the cell is a eukaryotic cell.

3. The method of claim 1 wherein the cell is a microbial cell.

4. The method of claim 1 wherein the cell is a bacterial cell.

5. The method of claim 1 wherein the cell is a yeast cell, a plant cell or a mammalian cell.

6. The method of claim 1 wherein the cell is a human cell.

7. The method of claim 1 wherein the polypeptide is a protein.

8. The method of claim 1 wherein the polypeptide is toxic to the cell.

9. The method of claim 1 wherein the polypeptide is toxic to the cell, wherein toxicity is determined by a reduction in cell growth rate by at least 1%, 10%, 50%, or 90% compared to growth rate of a corresponding wild type cell.

10. The method of claim 1 wherein the polypeptide is a protein that is toxic to the cell.

11. The method of claim 1 wherein the polypeptide is a protein that is toxic to the cell and wherein lack of the cell to produce (1) the one or more tRNA cognate to the one or more canonical codons or (2) the one or more release factors prevents expression of the foreign nucleic acid sequence into the polypeptide, thereby reducing toxicity to the cell of the polypeptide compared to a non-recoded cell of the same strain.

12. The method of claim 1 wherein the cell is grown to produce a population of cells including the foreign nucleic acid sequence.

13. The method of claim 13 wherein one or more tRNA cognate to the one or more unassigned codons are provided within the cell, and wherein the cell expresses the foreign nucleic acid sequence into the polypeptide.

14. A method for altering a cell comprising
providing a cell with a foreign nucleic acid sequence of canonical codons encoding a polypeptide,
wherein one or more canonical codon sequences within the foreign nucleic acid sequence are altered to produce one or more unassigned codons within the cell,
wherein presence of the unassigned codon reduces or prevents expression of the foreign nucleic acid sequence into the polypeptide,
wherein the unassigned codon is optionally located upstream of the nucleic acid sequence encoding the protein,
wherein the unassigned codon is optionally located in a promoter cognate to the foreign nucleic acid sequence, or
wherein the unassigned codon is optionally located within the open reading frame of the foreign nucleic acid sequence.

15. The method of claim 14 wherein the cell is a eukaryotic cell.

16. The method of claim 14 wherein the cell is a microbial cell.

17. The method of claim 14 wherein the cell is a bacterial cell.

18. The method of claim 14 wherein the cell is a yeast cell, a plant cell or a mammalian cell.

19. The method of claim 14 wherein the cell is a human cell.

20. The method of claim 14 wherein the polypeptide is a protein.

21. The method of claim 14 wherein the polypeptide is toxic to the cell.

22. The method of claim 14 wherein the polypeptide is toxic to the cell, wherein toxicity is determined by a reduction in cell growth rate by at least 1%, 10%, 50%, or 90% compared to growth rate of a corresponding wild type cell.

23. The method of claim 14 wherein the polypeptide is a protein that is toxic to the cell.

24. The method of claim 14 wherein the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

25. The method of claim 14 wherein one or more tRNA cognate to the one or more unassigned codons are provided within the cell, and wherein the cell expresses the foreign nucleic acid sequence into the polypeptide.

26. The method of claim 1 wherein the microbial cell is grown to produce a population of microbial cells including the foreign nucleic acid sequence.

27. A method for expressing a foreign nucleic acid sequence to produce a polypeptide in a cell, wherein the cell lacks expression of one or more tRNA cognate to one or more canonical codons or lacks expression of one or more release factors or otherwise lacks (1) one or more native genes operative to encode one or more tRNA cognate to one or more canonical codons within the foreign nucleic acid sequence or (2) one or more native genes operative to encode one or more release factors comprising
providing the cell with the foreign nucleic acid sequence,
optionally providing the cell with an expression plasmid or vector that is expressed (1) to provide the one or more tRNA cognate to the one or more canonical codons within the foreign nucleic acid sequence or (2) the one or more release factors, wherein the cell translates the foreign nucleic acid sequence to produce the polypeptide, or
optionally, wherein the cell is genetically modified to encode one or more tRNA cognate to one or more canonical codons within the foreign nucleic acid sequence under the influence of an inducible promoter and inducing the inducible promoter to begin translation of the foreign nucleic acid sequence to produce the polypeptide.

28. The method of claim 27 wherein the (1) one or more genes encoding one or more tRNA cognate to one or more canonical codons within the foreign nucleic acid sequence or (2) one or more genes encoding one or more release factors are under the influence of a constitutive promoter.

29. The method of claim 27 wherein the (1) one or more genes encoding one or more tRNA cognate to one or more canonical codons within the foreign nucleic acid sequence or (2) one or more genes encoding one or more release factors are under the influence of an inducible promoter.

30. The method of claim 27 wherein the expression plasmid expresses (1) the one or more tRNA cognate to the one or more canonical codons within the foreign nucleic acid sequence or (2) the one or more release factors at a first temperature but does not express (1) the one or more tRNA cognate to the one or more canonical codons within the foreign nucleic acid sequence or (2) the one or more release factors at a second temperature that is higher than the first temperature.

31. The method of claim 27 wherein the expression plasmid includes a resistance gene allowing selection of cells including the expression plasmid.

32. The method of claim 27 wherein the expression plasmid includes a counterselectable gene allowing counterselection of cells including the expression plasmid.

33. The method of claim 27 wherein the expression plasmid includes a nuclease cutting site.

34. The method of claim 27 wherein (1) one or more native genes encoding one or more tRNA cognate to one or more canonical codons within the foreign nucleic acid sequence or (2) one or more native genes encoding one or more release factors are rendered inoperative by CRISPR-Cas9 mediated genome editing or removal of the (1) one or more native genes encoding one or more tRNA cognate to one or more canonical codons within the foreign nucleic acid sequence or (2) one or more native genes encoding one or more release factors.

35. The method of claim 27 wherein the cell is grown to produce a population of cells including the foreign nucleic acid sequence.

36. The method of claim 27 wherein the cell is a eukaryotic cell.

37. The method of claim 27 wherein the cell is a microbial cell.

38. The method of claim 27 wherein the cell is a bacterial cell.

39. The method of claim 27 wherein the cell is a yeast cell, a plant cell or a mammalian cell.

40. The method of claim 27 wherein the cell is a human cell.

41. The method of claim 27 wherein the polypeptide is a protein.

42. The method of claim 27 wherein the polypeptide is toxic to the cell.

43. The method of claim 27 wherein the polypeptide is toxic to the cell, wherein toxicity is determined by a reduction in cell growth rate by at least 1%, 10%, 50%, or 90% compared to growth rate of a corresponding wild type cell.

44. The method of claim 27 wherein the polypeptide is a protein that is toxic to the cell.

45. The method of claim 27 wherein the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

46. An altered cell lacking expression of one or more tRNA cognate to one or more canonical codons or lacking expression of one or more release factors or otherwise lacking (1) one or more native genes operative to encode one or more tRNA cognate to one or more canonical codons or (2) one or more native genes operative to encode one or more release factors and comprising
a foreign nucleic acid sequence of canonical codons encoding a polypeptide.

47. The method of claim 46 wherein the cell is a eukaryotic cell.

48. The method of claim 46 wherein the cell is a microbial cell.

49. The method of claim 46 wherein the cell is a bacterial cell.

50. The method of claim 46 wherein the cell is a yeast cell, a plant cell or a mammalian cell.

51. The method of claim 46 wherein the cell is a human cell.

52. The method of claim 46 wherein the polypeptide is a protein.

53. The method of claim 46 wherein the polypeptide is toxic to the cell.

54. The method of claim 46 wherein the polypeptide is toxic to the cell, wherein toxicity is determined by a reduction in cell growth rate by at least 1%, 10%, 50%, or 90% compared to growth rate of a corresponding wild type cell.

55. The method of claim 46 wherein the polypeptide is a protein that is toxic to the cell.

56. The method of claim 46 wherein the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

57. An altered cell comprising
a foreign nucleic acid sequence of canonical codons encoding a polypeptide,
wherein the foreign nucleic acid sequence is recoded to include one or more unassigned codons replacing one or more target canonical codons,
wherein the unassigned codon is optionally located upstream of the foreign nucleic acid sequence encoding the protein,
wherein the unassigned codon is optionally located in a promoter cognate to the foreign nucleic acid sequence, or
wherein the unassigned codon is optionally located within the open reading frame of the foreign nucleic acid sequence.

58. The cell of claim 57 wherein the cell is a eukaryotic cell.

59. The cell of claim 57 wherein the cell is a microbial cell.

60. The cell of claim 57 wherein the cell is a bacterial cell.

61. The cell of claim 57 wherein the cell is a yeast cell, a plant cell or a mammalian cell.

62. The cell of claim 57 wherein the cell is a human cell.

63. The cell of claim 57 wherein the polypeptide is a protein.

64. The cell of claim 57 wherein the polypeptide is toxic to the cell.

65. The cell of claim 57 wherein the polypeptide is toxic to the cell, wherein toxicity is determined by a reduction in cell growth rate by at least 1%, 10%, 50%, or 90% compared to growth rate of a corresponding wild type cell.

66. The cell of claim 57 wherein the polypeptide is a protein that is toxic to the cell.

67. The cell of claim 57 wherein the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.

68. An altered cell lacking expression of one or more tRNA cognate to one or more canonical codons or lacking expression of one or more release factors or otherwise lacking (1) one or more native genes operative to encode one or more tRNA cognate to one or more canonical codons within a foreign nucleic acid sequence encoding a polypeptide or (2) one or more native genes operative to encode one or more release factors comprising
the foreign nucleic acid sequence,
an expression plasmid or vector encoding the one or more tRNA cognate to the one or more canonical codons within the foreign nucleic acid sequence or (2) the one or more release factors.

69. The cell of claim 68 wherein the cell is a eukaryotic cell.

70. The cell of claim 68 wherein the cell is a microbial cell.

71. The cell of claim 68 wherein the cell is a bacterial cell.

72. The cell of claim 68 wherein the cell is a yeast cell, a plant cell or a mammalian cell.

73. The cell of claim 68 wherein the cell is a human cell.

74. The cell of claim 68 wherein the polypeptide is a protein.

75. The cell of claim 68 wherein the polypeptide is toxic to the cell.

76. The cell of claim 68 wherein the polypeptide is toxic to the cell, wherein toxicity is determined by a reduction in cell growth rate by at least 1%, 10%, 50%, or 90% compared to growth rate of a corresponding wild type cell.

77. The cell of claim 68 wherein the polypeptide is a protein that is toxic to the cell.

78. The cell of claim 68 wherein the polypeptide is a protein that is toxic to the cell and wherein lack of the cell to produce (1) one or more tRNA cognate to the one or more unassigned codons or (2) the one or more release factors prevents expression of the foreign nucleic acid sequence into the polypeptide, thereby reducing toxicity to the cell of the polypeptide compared to a non-recoded cell of the same strain.
